(19) United States
(12) Patent Application Publication
(10) Pub. No.: US 2025/0340956 A1
(43) Pub. Date: Nov. 6, 2025

(54) AUTOMATED SYSTEM AND METHODS FOR DISEASE DETECTION

(71) Applicant: FLORIDA ATLANTIC UNIVERSITY BOARD OF TRUSTEES, Boca Raton, FL (US)

(72) Inventor: Waseem ASGHAR, Boca Raton, FL (US)

(21) Appl. No.: 18/007,593

(22) PCT Filed: Jun. 1, 2021
(86) PCT No.: PCT/US2021/035203

§ 371 Date: Dec. 1, 2022

Related U.S. Application Data
(60) Provisional application No. 63/033,253, filed on Jun. 2, 2020.

(51) Int. Cl. C12Q 1/70; B01F 33/30; B01F 33/452; B01L 3/00; C12Q 1/6844

(52) U.S. Cl. CPC C12Q 1/701; B01F 33/30; B01F 33/452; B01L 3/502761; C12Q 1/6844; B01L 2200/0668; B01L 2200/16; B01L 2300/0663; B01L 2300/1805; B01L 2400/0688

(57) ABSTRACT

An automated platform for the detection of a virus, bacteria, and/or other organism of interest from a clinical sample and a method of isolation, purification, and amplification of viruses from clinical samples using a Loop-Mediated Isothermal Amplification (“LAMP”) method on the automated platform is described herein.

### CROSS-REFERENCE TO RELATED APPLICATIONS

This application is a United States Nation Phase entry under 35 U.S.C. § 371 of International Application No. PCT/US2021/035203, filed Jun. 1, 2021, which claims the benefit under 35 U.S.C. § 119(e) of Provisional Application Ser. No. 63/033,253 filed on Jun. 2, 2020 entitled AUTOMATED SYSTEM AND METHODS FOR DISEASE DETECTION and whose entire disclosure is incorporated by reference herein.

### FIELD OF INVENTION

This invention relates to an automated microfluidic platform for point-of-care (“POC”) and home settings that detects viruses, bacteria, and/or other organism of interest from clinical samples with high specificity and sensitivity, eliminating human intervention at any step after sample loading.

### REFERENCE TO SEQUENCE LISTING

A Sequence Listing conforming to the rules of WIPO Standard ST.25 is hereby incorporated by reference. Said Sequence Listing has been filed as an electronic document via Patent Center in ASCII format encoded as TXT. The electronic document, created on Jul. 13, 2023, is entitled “11605-022US1_ST25.txt”, and is 4,096 bytes in size.

### BACKGROUND OF THE INVENTION

According to the World Health Organization (WHO), every year more than 10 million deaths are caused by infectious diseases worldwide. For example, Human Immunodeficiency Virus (HIV) alone has caused more than 25 million deaths and has become one of the most devastating pathogens in human history. Worldwide, about 1 in 4 of people who have contracted the virus are unaware of their HIV status. More examples of infectious diseases are flavivirus infections, such as dengue, zika, chikungunya, and yellow fever.

Around 3.6 billion people, nearly half the world's population, live in flavivirus endemic areas. The high transmission potential of these viruses, combined with poor surveillance in resource-limited settings, can result in epidemics. A number of challenges contribute to the higher rates of viral transmission. People who do not know that they are infected with some infectious virus cannot take advantage of specialized care and may unknowingly infect others. Additionally, the socioeconomic issues associated with poverty including limited access to high-quality health care directly and indirectly increase the risk for viral infection.

There are many diseases spread by mosquitoes which includes Zika, dengue, yellow fever, and chikungunya infections. Zika virus (ZIKV) infection is associated with neurological complications such as Guillain-Barre syndrome (GBS), meningoencephalitis, acute myelitis in adults and microcephaly in infants. According to the Centers for Disease Control and Prevention (CDC), from the year 2015 to 2018, 5,304 ZIKV cases were reported in the United States and during the same time, 36,522 ZIKV cases were observed in US territories.

During the acute phase of viral infection (the first two weeks), there is a high viral load present in blood and increased chances of spreading the infection to other people. While the main mode of ZIKV transmission is mosquitos, the other transmission modes include sexual transmission, infants born to mothers with established ZIKV infection, breast milk, saliva, blood transfusion and needlestick. The symptoms of ZIKV are closely associated with other mosquito-borne vector infections such as dengue, yellow fever, and chikungunya virus which include headache, rash, fever, and joint pain. All of these viruses share similar symptoms making it difficult to detect ZIKV in the patient. Unfortunately for many infectious agents, there is no portable detection platform available during the acute phase of infection that can be performed at POC settings.

For all these diseases, improved and automated diagnostic methods appropriate for resource-poor settings and home settings are required for the early detection and the rapid response to contain viral outbreaks and reduce their associated morbidity and mortality.

Currently available molecular diagnostic assays that utilize reverse transcription quantitative polymerase chain reaction (RT-qPCR) are highly specific, sensitive, and can distinguish between various viral infections. However, these assays are complex, require multiple labor-intensive steps, and need costly and bulky equipment (e.g. thermocycler). Hence, they are not suitable for testing at POC settings.

Further current molecular detection systems (e.g. PCR) require extensive sample preparation, viral RNA/DNA isolation and purification steps that are manual and labor-intensive and cannot be performed at resource-constrained and POC settings.

There are several diagnostic methods developed for ZIKV detection such as lateral flow assays (LFAs), enzyme-linked immunosorbent assay (ELISA), IgM antibody capture enzyme-linked immunosorbent assay (MAC-ELISA), and reverse transcriptase polymerase chain reaction (RT-PCR).

RT-PCR is a conventional ZIKV specific method that remains the gold-standard for disease detection from the patient sample. However, RT-PCR is time consuming and requires trained personnel as well as expensive equipment such as thermocycler. This poses a challenging issue for people living in areas where medical facilities are minimal and access to laboratory services is difficult.

Additionally, pure RNA isolated from blood, urine or plasma is used for the detection of the virus, impurities from the raw sample can inhibit the reaction and can also give false negative results.

Nearly two decades ago, Loop-Mediated Isothermal Amplification (“LAMP”) method was reported and was capable of amplifying DNA and RNA at an isothermal temperature. It is a quick, robust and specific method that amplifies the target at a fixed temperature that usually ranges between 65-74° C. with the help of 4-6 set of primers. LAMP eradicates the requirement of different temperature cycling making it the better amplification method over PCR for the low-cost POC diagnostics.

For disease detection, treatment validation and outcome, POC diagnostics plays an important role in expediting the detection of the disease in resource constrained areas.

Previously, significant efforts have been done for the development of POC ZIKV diagnostics. All of these developed methods have several limitations such as complex chip assembly, manhandled processing steps, air-drying membrane before introducing the LAMP reagents, and equipment (e.g.—smartphone) required for result interpretation. Furthermore, the colorimetric detection method in at least one of the studies demonstrated false positive results due to non-specific binding.

Accordingly, it is desired to provide a new, portable, automated system that is capable of rapidly and accurately recognizing viruses by RNA/DNA isolation from a human sample.

It is also desired to provide a simple, reliable, and cost-efficient platform that provides high sensitivity and specificity for ZIKV detection without the access of trained technicians, expensive equipment, or special facilities including electricity.

Al references cited herein are incorporated herein by reference in their entireties.

### BRIEF SUMMARY OF THE INVENTION

A first aspect of the invention comprises a fully automated, true sample-in-answer-out multi-chamber disposable device integrated with magnetic actuation platform that can perform automated RNA/DNA isolation, washing, purification, and isothermal amplification all on a single chip.

A second aspect of the invention is an automated lab-on-a-chip microfluidic platform that detects ZIKV from human blood plasma with high specificity and sensitivity eliminating human intervention at any step after sample loading, and unifies multiple steps on the same platform.

The overall invention is a portable pathogen detection platform for POC and home settings that can (i) specifically and simultaneously detect multiple viruses including DENY, ZIKV, HIV, coronavirus, and HCV from clinical samples (blood, serum, plasma, swab, saliva, or urine) (ii) be highly sensitive within the clinical range, (iii) rapid, and (iv) fully automated.

### BRIEF SUMMARY OF THE DRAWINGS

FIG. 1A is a representation of fully assembled three-layer PMMA chip;

FIG. 1B is a representation of the independent chambers and valving chambers of the chip filled with buffers and the reagents;

FIG. 1C is an illustration of overall process illustration of benchtop nucleic acid extraction from plasma with viral nucleic acid (“NA”) kit;

FIG. 2 depicts specific functions of the independent chambers of microfluidic chip;

FIG. 3 depicts the assembled automated microfluidic set-up;

FIG. 4 is an amplification plot of the benchtop assay with different ZIKV concentrations per reaction;

FIG. 5 is amplification curve of the ZIKV spiked plasma samples (102 to 107 copies per mL) pertinent to clinical range;

FIG. 6 is an RT-LAMP amplification plot for the 2 cultured and 2 clinical plasma ZIKV and 1 clinical plasma Dengue samples;

FIG. 7 is a snapshot of the sequences aligned in Clustal W, wherein the asterisk sign (*) shows the conserved nucleotide in all the sequences. Five nucleotides marked with green color out of which 2 are circled to highlight the vertical mismatch nucleotide between the chosen ZIKV sequences; Sequences in FIG. 7 include SEQ ID NO: 7, 8, 9, and 10;

FIG. 8 are baseline subtracted amplification points of the ZIKV target representing change in the fluorescent intensity over the time and the standard curve;

FIG. 9 is a grey value plot of the off-chip saturated reaction measured by converting RGB pixel to mean grey intensity using ImageJ (Defined threshold value=175);

FIG. 10 depicts a baseline subtracted amplification points of the ZIKV target/mL isolated from the plasma indicating change in the fluorescent intensity over the interval and the average trendline;

FIG. 11 is a graphical representation of the 46 minutes temperature readings for the heater designed to heat the sensor chamber solution. The sensor recorded the reading every two seconds;

FIG. 12 is a grey value plot of the on-chip saturated reaction measured by converting RGB pixel to mean grey intensity using ImageJ (Defined threshold value=175).

### DETAILED DESCRIPTION OF PREFERRED EMBODIMENTS OF THE INVENTION

In this invention, to enable point-of-care testing, a microfluidic chip containing 4 different chambers for different processed involved in LAMP based detection is utilized which combines the isolation, purification, and amplification steps on the same platform. The chip retains the liquid inside the channels and avoids mixing of the reagents and assisted movement of magnetic beads for the point-of-care testing. The hydrophobic interaction in the aqueous chambers holds the fluid and the curvature of valving chamber provides less turbulence that facilitates the easy flow of the magnetic beads.

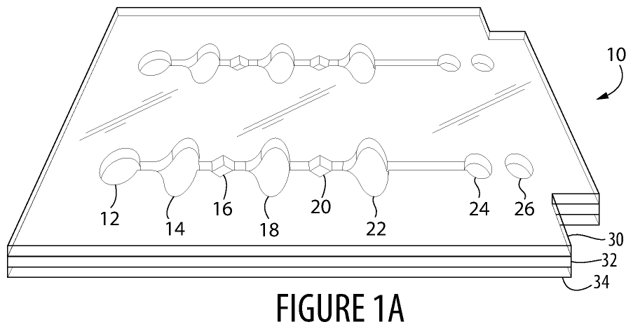

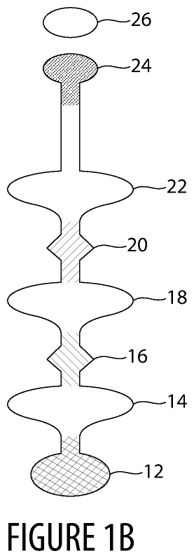

In certain embodiments, the automated platform may include a disposable microfluidic chip 10 (FIG. 1A), a magnetic actuation platform 48, a surface heater 64; and a laptop or other computing device 62 to regulate the automatic magnetic actuation and control the heater's temperature.

The developed disposable microfluidic chip 10 has multiple aqueous chambers separated by oil chambers which work as valves. To automate the sample prep, amplification, and detection, a magnetic actuation-based system can precisely move the magnetic beads from one well to the other in an automated fashion without mixing aqueous chambers on a single device.

The device can have all the reagents stored in the dry form that can be reactivated with a buffer before use.

In certain embodiments, the microfluidic chip 10 is assembled in three layers which may include a top layer 30 which may be made using of poly (methyl methacrylate) (PMMA) at a thickness of 750 μm, a middle layer 32 which may be made using poly (methyl methacrylate) (PMMA) at a thickness of 1.5 mm; and a bottom layer 34 which may be made using poly (methyl methacrylate) (PMMA) at a thickness of 750 μm (FIG. 1B).

In certain embodiments, the layers are attached using double sided adhesive tape.

In certain embodiments, each microfluidic chip 10 may include a plurality of independent aqueous chambers, including an oval shaped inlet chamber 12, at least one diamond-shaped washing buffer chamber; preferably a first washing buffer chamber 16 and a second washing buffer chamber 20, an amplification chamber 24, at least one elliptical shaped valving chamber containing mineral oil; preferably a first valving chamber 14, a second valving chamber 18, and a third valving chamber 22, and an unconnected oval-shaped sensor chamber 26.

In certain embodiments, the top layer 30 contains two pipette inlets which may be 0.4 mm diameter above each chamber. One inlet may be used to discharge the fluid into the chip and another may liberate the air out of the chamber.

In certain embodiments, for example, the microfluidic chip 10 may include, in total, eight independent chambers.

In certain embodiments, the microfluidic chip 10 may include two washing buffer chambers; preferably a first washing buffer chamber 16 and a second washing buffer chamber 20.

In certain embodiments, the microfluidic chip 10 may include three elliptical shaped valving chambers; preferably a first valving chamber 14, a second valving chamber 18, and a third valving chamber 22.

In certain embodiments, the unconnected oval-shaped sensor 26 chamber may be carefully separated from other chambers to prevent the precipitate formation of the buffers due to the heating effect.

In certain embodiments, the mineral oil has a viscosity of 15 cSt.

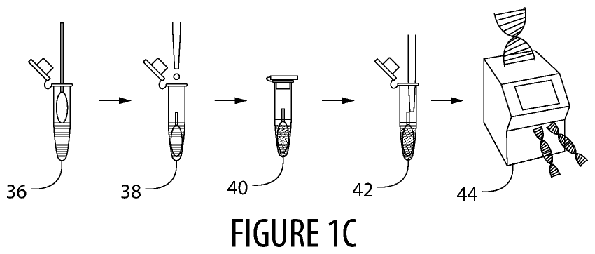

In certain embodiments, the magnetic actuation platform 52 (FIG. 3) is executed by at least one small magnet 60 which may beS mm-diameter neodymium. This at least one magnet(s) may be enclosed with a stepper motor 54 and able to move bidirectionally via stepper motor linear slide rails 56 which are connected to the stepper motor 54 by a power output wire 58 (FIG. 3).

In certain embodiments, the magnetic actuation controller 48 automatically moves the microparticles through the microfluidic chip 10. The magnetic actuation s platform 52 may include the stepper motor 54 and the stepper motor linear slide rails 56, and at least one, preferably 2, 5 mm neodymium magnet(s) 60. The microfluidic chip 10 may be aligned with the at least one magnet(s) 60 by a 3-D printed enclosure and the at least one magnet(s) 60 is held in place by a carriage 46 on the stepper motor linear slide rails 56. Additional electronic components may include a microprocessor, a stepper motor driver, and power supply circuits.

In certain embodiments, the magnetic actuation platform 52 may be coordinated by, for example, a printed circuit board shield input for software control and integration. The circuit board may control the magnetic bead's 60 movement from one chamber to another.

In certain embodiments, incubation time in each chamber may be controlled, for example, by a g-code scripted in python.

In certain embodiments, human-readable commands may be sent from a laptop or other computing device 62 to a microprocessor through a serial interface. The microprocessor may translate the human-readable commands into stepper motor 54 driver commands and may control the stepper motor linear slide rails 56 through the driver.

The inventors have surprisingly determined that the magnetic beads 60 may be, for example, actuated by the stepper motor 54 at 25 mm per second and may allow for change in direction for sufficient bead mixing in each chamber as the pellet is actuated in the forward and backward directions within the microfluidic chip 10.

The inventors identified four commands that are required for the actuation sequence of preferred embodiments. The software parses the sequence of commands from a file and sends them to the actuator, while ensuring that all commands are executed. Using this method, a change in microfluidic chip geometry can be accommodated by using a different command file.

To enable on-chip heating capabilities, the inventors have developed an automated circuit board based temperature control system 68 to strictly control the temperature of the reagents enclosed in the amplification chamber 24 on the microfluidic chip 10. The sensor chamber 26, which contains a sensor, and the amplification chamber 24 may be filled with the same reagents and a surface heater 64 may be attached on top of both chambers.

In certain embodiments, the developed system may have an in-built heater to control the temperature required for isothermal amplification.

In certain embodiments, to enable on-chip heating capabilities, an automated circuit board based temperature control system 68 may control the temperature of the reagents enclosed in the amplification chamber 24 of the microfluidic chip 10.

In certain embodiments, no external power source is required.

In certain embodiments, the automated platform is highly scalable and multiple samples may be tested simultaneously.

For certain the clinical fluids, such as blood, a filter may be integrated before the inlet chamber 12 of the device that may isolate blood cells from the sample so that only plasma will flow to the inlet chamber 12 for RNA/DNA isolation.

In certain embodiments, once the target sample is introduced to the inlet chamber 12, the developed automated platform may motorize the target isolation, purification, and amplification for the disease detection on the chip. Change of color upon the presence of the ZIKV target in the amplification chamber 24 may be observed due to the colorimetric properties of leucocrystal violet (“LCV”) dye.

The on-chip results from this automated assay may show, for example, its sensitivity by showing positive results with the plasma having a minimum clinical range of target (10² plaque-forming unit (PFU)/mL) found in ZIKV infected patient.

The invention will be illustrated in more detail with reference to the following Examples, but it should be understood that the present invention is not deemed to be limited thereto.

### EXAMPLES

### Materials and Methods

Materials. A k-type thermocouple 70 sensor reads the actual temperature of the reagent in real-time by interfacing with the MAX6675 module (Maxim integrated, USA) using the surface heater attached on top of both chambers. A microcontroller (for example, an Arduino) controls the 1.98 watts 2 cm×2 cm rectangular-shaped ultra-thin nano carbon flexible heater (TSA(C) 0200020eR12.6, Pelonis Tech Exton, PA) by controlling the MOSFET gate signals. The microcontroller powers the heater (5V), therefore no external power source is required. The “on” red LED visualizes the heater operation by lighting the LED when it is heating the reagents in the sensor chamber and turned “off” when the feedback temperature is higher than the set temperature. The k-type thermocouple sensor controls the temperature of the amplification chamber which was set at 70° C.±2° C.

Example 1: Automated platform integration & on-chip sample processing. Human plasma samples were taken from a healthy individual and spiked with the different concentrations of ZIKV target (10, 102, 103, 104, and 108 target copies/mL) relevant to the clinical range of the patients (102 to 106 PFU/mL) to load in the inlet chamber. The negative controls were 108 copies/mL HIV target spiked in human plasma and unbiased plasma sample collected from a healthy person. For the on-chip testing, the amplification chamber was filled with LavaLAMP RNA Master Mix, ZIKV specific primers, elution buffer and leucocrystal violent (“LCV”) for the colorimetric detection. DNA is amplified using Loop-Mediated Isothermal Amplification (“LAMP”), which uses 4-6 primers recognizing 6-8 distinct regions of target DNA. The LAMP reaction reagents, which include LavaLAMP RNA Master Mix, LCV dye, and LAMP primers, for the amplification chamber were formulated before running the experimentation. The pipette inlets of the amplification chamber and sensor chamber were sealed with epoxy glue (Loctite epoxy) to avoid the evaporation of the master mix which dried within 5 minutes. Table 1, below, lists the Zika RT-LAMP primer sequences alongside the concentration.

TABLE 1
List of Zika RT-LAMP primer sequences
alongside the concentration.

| Primers | | concentration |
|---|---|---|
| Name | Sequence 5′ to 3′ | (μM) |
| F3 | CTGGTTGGTTCACAAG | 0.2 |
| | (SEQ ID NO: 1) | |
| B3 | CAGAGCTCCAGCAAG | 0.2 |
| | (SEQ ID NO: 2) | |
| FIP | GTGTGGAGTTCCGGTGTCT- | 1.6 |
| | AGTGGTTCCACGACATTCC | |
| | (SEQ ID NO: 3) | |
| BIP | CTGGTAGAGTTCAAGGACG | 1.6 |
| | CA-TGCTCCTTCTTGACTC | |
| | CCTA | |
| | (SEQ ID NO: 4) | |
| LF | CCCAGCGTGCCAAGGTAAT | 0.4 |
| | (SEQ ID NO: 5) | |
| LB | CATGCCAAAAGGCAAACTG | 0.4 |
| | TC | |
| | (SEQ ID NO: 6) | |

The first step (plasma sample for the inlet chamber) was carried out in an Eppendorf tube (0.5 mL tubes) to ensure accuracy. In the Eppendorf tube, 20 μl of proteinase K was added to 100 μl of plasma followed by the addition of 95 μl of lysis and binding buffer. The tube was incubated for 2-3 minutes at room temperature after which 45 μl of isopropanol and 15 μl magnetic beads were added and incubated on the rocker for about 5-7 minutes. While the sample incubated on the rocker, with the pipette through pipette inlets the chip was loaded with a washing buffer 1 (1:1-buffer 1:nanopure water) in the first buffer chamber and buffer 2 in the second buffing chamber. These washing buffers are composed of TE buffer+1 M NaCl+ethonaol, pH 7.0-7.5. Other commercial washing buffers may also be used. After loading these chambers, the mineral oil was added to the valving chambers and in the end, the plasma sample mixed with buffers and magnetic beads was introduced into the inlet chamber.

Immediately after the chip loading, the surface heater was attached on top of the amplification chamber and sensor chamber. The heater was turned “on” 3 minutes before the start of magnetic actuation and once the target was eluted in the amplification chamber, magnetic beads were moved back to the second washing buffer chamber. The heater was kept “on” for an additional 30 minutes for the LAMP amplification process and after the isothermal incubation, the results were visually observed. The volume of each chamber and incubation time of the magnetic beads is presented in Table 3, below.

TABLE 2
Chip volume and magnetic actuation time.

| | | | Total time for |
|---|---|---|---|
| | | | magnetic |
| | | | actuation- 9 |
| | | | minutes 20 |
| Chambers | Buffers/reagents | Total volume | seconds |
| Chamber 1 | Plasma sample + | 100 μl | 3 minutes |
| | magnetic beads | | |
| Chamber 2 | Washing buffer 1 | 45 μl | 1 minutes 40 |
| | | | seconds |
| Chamber 3 | Washing buffer 2 | 45 μl | 1 minutes 40 |
| | | | seconds |
| Chamber 4 | Elution buffer | 55 μl | 3 minutes |
| | (comprised of 10 mM | | |
| | tris HCl, pH 8.0-8.5) + | | |
| | master mix + Primers + | | |
| | LCV (leucocrystal | | |
| | violet) | | |
| Valving | Oil | 154 μl | |
| chambers | | | |

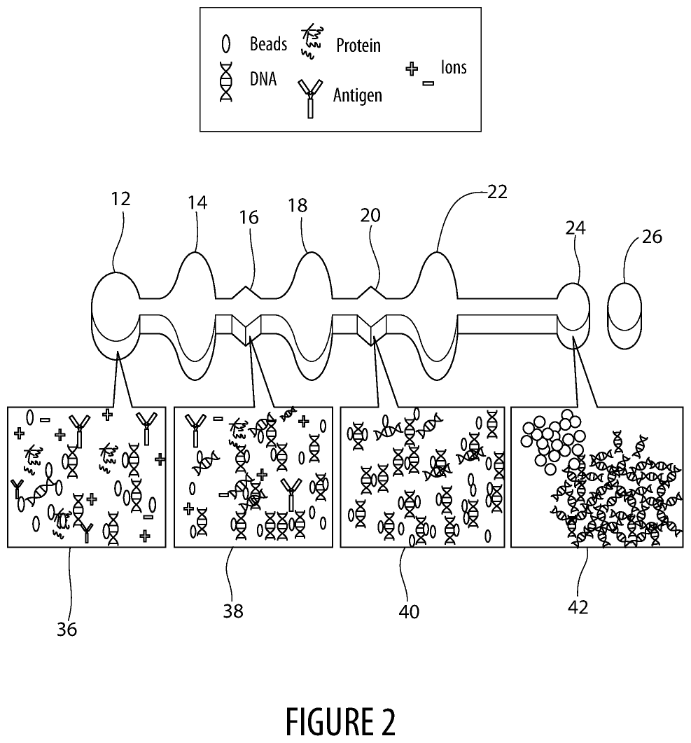

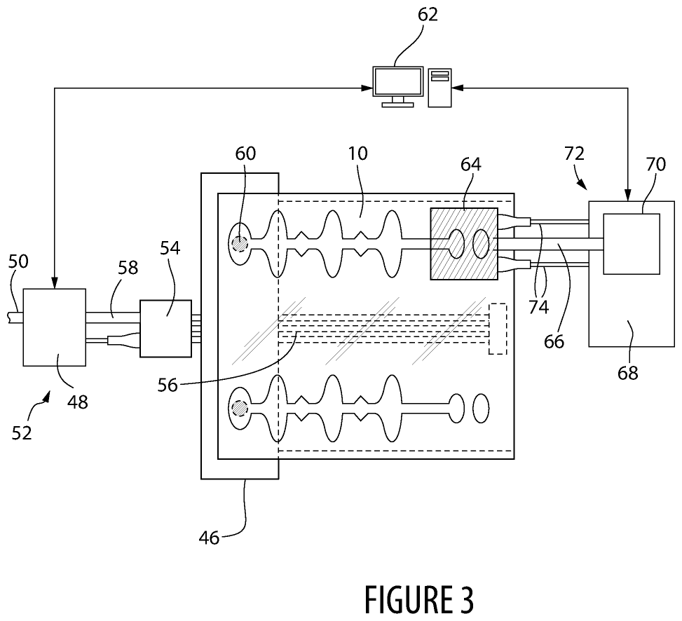

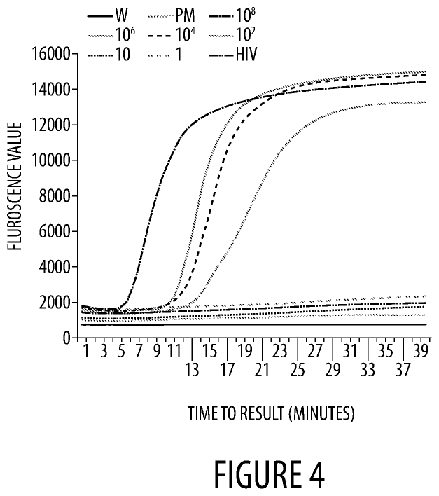

Example 2: Benchtop LAMP Amplification Results. Isothermal amplification and less sensitivity of LAMP towards common amplification inhibitors make it a better choice for healthcare infrastructure in resource-limited areas and also overcomes the main limitations (cost, sensitivity, etc.) of the current gold standard PCR technique. However, the inadequacy of using several primers in the LAMP leads to the primer-dimer formation (non-target amplification) which can result in false positives. The reaction carried out with designed ZIKV primers and Master Mix to eliminate the non-target amplification indicated no sign of amplification. The collected fluorescent data showed no change in fluorescent intensity over the period which stipulates that designed primers would bind exclusively to the ZIKV target. Simultaneously, the primers amplified the synthetic ZIKV target and an abrupt increase in the fluorescent value was seen in the reactions holding different ZIKV concentrations. The lowest limit of detection observed was 1 ( ) 2 DNA copies/reaction and the time to result (TTR) for the amplification reaction carrying 10², 10⁴, 10⁶· and 10⁸ copies/reaction fall anywhere between 5 to 15 minutes. At the same time, no increase in the fluorescent signal was observed in the negative control reaction carrying HN target, this disclosed the primer's specificity towards the ZIKV target. FIG. 4 represents the amplification plot of the benchtop experiment for specificity, sensitivity, and the primer dimer formation evaluation.

In the LAMP reaction, the real-time fluorescent signals are not always exponential because during the amplification process, the DNA segments are extended, and simultaneously short segment are also produced. 1% gel electrophoresis further confirmed these LAMP off-chip results. The long sharp bands of the ZIKV amplicons stained with loading dye in the wells could be seen exhibiting amplification confirmation and no band formation spotted in HN, PM, and water wells, validating the specificity of these primers.

The inventors also unexpectedly examined a visual detection method that eliminated the need for fluorescence excitation equipment, making the platform more user-friendly. LCV is generally colorless, however, in the presence of dsDNA, the LCV interrelates with the major grooves of the dsDNA thereby converting LCV into crystal violet. This imparts a violet color to the solution for colorimetric visual observation. After the isothermal amplification the reactions that exhibited a dark crystal violet coloration show that the ZIKV target amplified. On the other hand, the faint blue coloration in tubes signifies that the LAMP reaction did not proceed, and no amplification occurred. Notably, the target detection sensitivity remained as before i.e. 10² DNA copies/reaction.

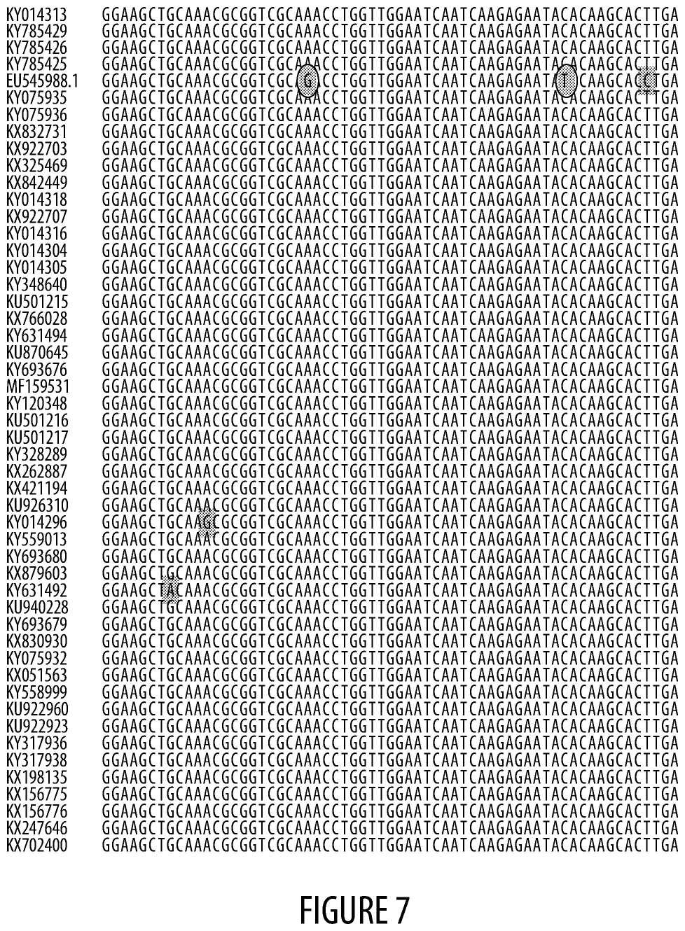

Further, the original RGB image was analyzed using ImageJ and the intensity was calculated by converting RGB pixels to grey value. FIG. 9 corresponds to the grey intensity value of the resultant products. The amplification reaction is saturated therefore the intensity of the color was independent of the initial target concentration. The defined threshold value (175 a.u.) clearly depicts the difference between the negative reactions (W, PM, HIV and 10 copies/reaction) and the positive reactions (102, 104, 106 and 108 copies/reaction). This test indicates that the naked-eye test is accurate and amplified the specific ZIKV target in LAMP reaction.

Example 2: Results of Spiked Samples & Clinical Samples. To present a proof-of-concept of the designed assay, the synthetic target was spiked into the human plasma samples with clinical viremia range (10² to 10⁷ copies/mL). The target DNA was isolated from the human plasma with the abovementioned viral NA kit followed by the LAMP. The low pH of the buffers facilitates target binding to the magnetic beads and a high pH of elution buffer at a high temperature of 70° C. releases the target into the elution buffer. Isothermal fluorescent data obtained from the thermocycler showed a rise in fluorescent signals with the reactions containing the ZIKV target extract from the human plasma. The lowest limit of detection observed is 10² copies/mL. Neither the increase in fluorescent intensity of the HIV target (extracted from plasma) was seen nor the primer-dimer formation was observed during the entire time of incubation.

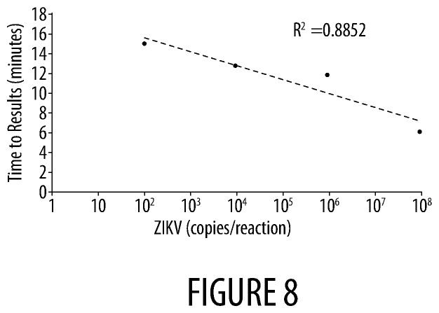

The results indicate that the viral NA kit is highly capable of isolating the target from plasma with excellent capture efficiency, and the designed LAMP primers are very efficient in amplifying the low quantity of ZIKV. Moreover, this also validates that the buffers used from the kit remove the unwanted components from the plasma which could possibly interfere with the LAMP. FIG. 10 represents the linear trendline with R2=0.9366, certifies the efficiency of the LAMP reactions carrying different concentration of targets isolated from plasma samples. Inventors successfully verified the results with 1% agarose gel electrophoresis in which the sharp bands correspond to ZIKA amplification in the wells containing the ZIKV target.

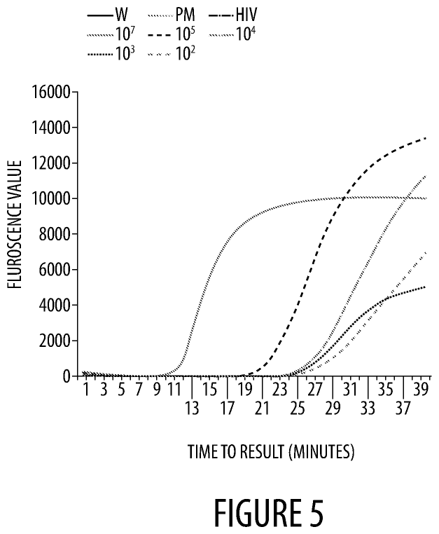

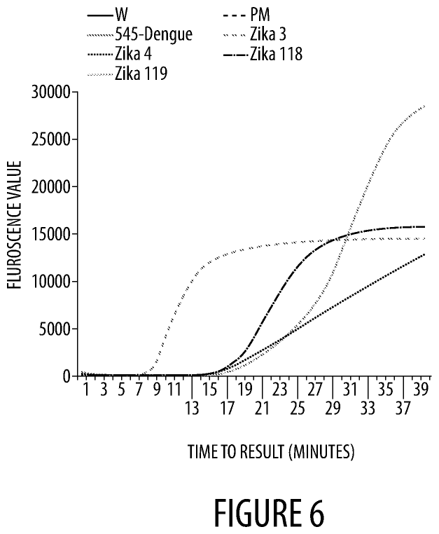

All the flaviviruses contain single positive-strand RNA, but the RNA based detection methods come with the challenge of RNA degradation during the extraction process. Inventors tested the viral NA kit and the designed primers against the clinical virus samples. The real-time fluorescent data obtained from the RT-LAMP reaction (FIG. 7A) showed a clear amplification trend with the Zika 3, Zika 4, Zika 118, and Zika 119 (ZIKV sample). Zika 3-cultured sample showed an early sign of amplification at TTR 7 minutes.

Zika 4, Zika 118 and Zika 119 samples showed the rise in the signal after 15 minutes of incubation, however, the saturated fluorescent amplification signals were achieved within 30 minutes. The viral quantification of the clinical plasma sample was unknown; however, amplification signal validates the detection sensitivity of the assay in terms of “yes-or-no” at different TTR.

The RT-LAMP exponential curve is often unprecise therefore, difference in the amplification efficacy and TTR was observed in the cultured samples. Furthermore, no rise in fluorescent signals with 545-dengue (Dengue sample) and PM reactions was observed that eradicated the risks of cross-reactivity with these novel designed primers. These results were verified by 1% gel electrophoresis, where the band formation illustrated no inhibitory effect of viral NA kit and primers with the ZIKV samples, and the dengue sample did not show any sign of amplification further verifying the primer's specificity towards ZIKV.

The experimental results of clinical virus samples proved that the developed assay and reagents used are compatible not only with DNA but also with RNA by maintaining its integrity for the virus detection. These experiments validated the capability of the designed primers to specifically amplify ZIKV RNA targets.

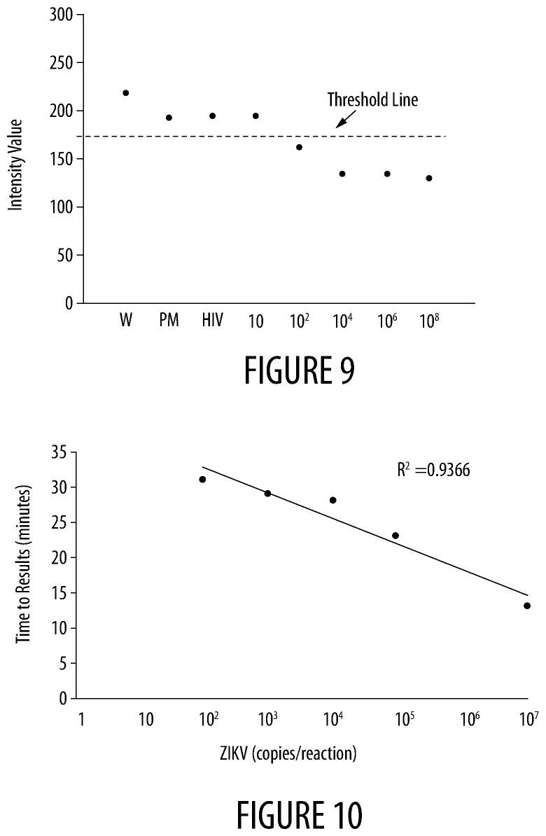

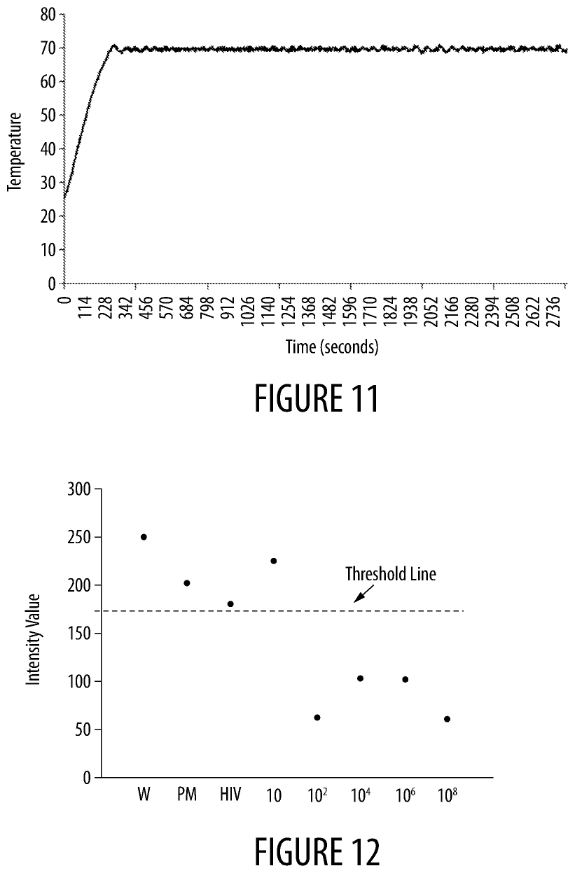

Example 3: Microfluidic Chip & Automated platform. The optimal temperature for LAMP amplification ranges from 68-74° C., therefore, to maintain the ideal on-chip amplification conditions, the temperature was chosen 2° C. higher than the off-chip experimental temperature used in the thermocycler. Additionally, the elution of nucleic acid content from the magnetic beads takes place at 70° C., thus, the heater was turned “on” 3 minutes before the start of magnetic actuation to ensure by the time the beads reached chamber 4 to elute the target, the chamber acquired the required temperature. The temperature display (FIG. 11).) shows the measured feedback every 2 seconds. The graph illustrates approximately after 4 minutes the chamber reagents gains the set temperature of 70° C. which is optimum for the elution of the target. The feedback temperature control system efficiently controls the temperature for all the phases which supports accurate target elution and amplification for the ZIKV detection. Additionally, to avoid interference with colorimetric results, the magnetic beads are moved back to the second washing buffer chamber after the target elution.

Example 4: On-Chip Spiked Plasma Results. To demonstrate the capability of visual detection of ZIKV using the developed system, the automated microfluidic platform test was conducted in the laboratory. The spiked ZIKV plasma samples containing 108, 104, 103 and 102 target copies/mL showed positive results of the target intensification by changing the color of the reagents in the amplification chamber. The presence of dsDNA changed the color to violet, which showed the successful amplification of the ZIKV target.

The results revealed that the device has optimal settings for the target isolation and purification. The lowest clinical viremia range of a ZIKV infected patient is 102 PFU/mL and this setup achieves the sensitivity of 102 copies/mL for target detection. At the same time, the on-chip negative control HIV spiked sample assay did not change color in the amplification chamber, which implied that the assay was highly specific to the ZIKV target. Similarly, on-chip negative control unbiased plasma results also eliminated the odds of the false-positive outcome by not showing the color change, showing no primer dimer formation.

Furthermore, for the sensitivity test with an automated microfluidic platform, the target copies were lowered to 10 ZIKV copies/mL in the plasma. No change in the color of amplification chamber demonstrated negative results, indicating the set up was unable to detect the ZIKV target with such a low target concentration in the plasma. The plasma sample volume used for this set-up was 100 μl for each run.

The multistep target extraction process, which is unified on the platform, has proven to avoid contamination for the downstream amplification process. The magnetic beads are compatible to the LAMP reaction reagents therefore the elution and amplification can take place in the same chamber making this microfluidic chip less complex. The successful on-chip amplification of the ZIKV target from the plasma sample is attributed by several factors such as use of small-scale volume of the buffers and reagents for the operations, capture efficiency of the magnetic beads, and compatibility of the magnetic beads with the LAMP reagents/polymerase. Moreover, the visual and non-inhibitory effect of the dye provided a high on-chip sensitivity. Furthermore, on-chip positive amplification reaction (102, 103, 104 and 108 target copies/mL) showed significantly lesser value than 175 (threshold value) and negative amplification reactions showed value higher than threshold. The on-chip and off-chip grey intensity values follow the same trend hence validating accuracy and specificity of the primers, reagents and the colorimetric dye on the microfluidic chip.

Before the incubation, the color of the amplification chamber was light blue and nearly colorless. After the beads eluted the target into the amplification chamber, the chamber was incubated for 30 minutes at 70° C.±2° C. to allow LAMP amplification. The color clearly changed in amplification chamber of the chip with spiked ZIKV plasma samples (except for the sample containing 10 copies/mL), hence, signifying the sensitivity of the automated set-up. No color change was observed with negative controls, indicating the specificity of the set-up towards ZIKV.

The inventors have developed an automated microfluidic chip-based LAMP assay that combines the isolation, purification, and the amplification steps on the same platform and enables the visual detection of the ZIKV from human plasma within 40 minutes. The entire ZIKV diagnosis procedure is executed inside a disposable microfluidic chip. All the components and reagents associated with the assay are optimized to provide highly specific and qualitative results without any human involvement after sample loading. The experimental confirmation of the test against negative controls, including dengue, HIV, and unbiased plasma from healthy humans verifies the specificity of the primers towards ZIKV, which addresses one of the main issues of false-positive (cross-reactivity) results faced in POC diagnostics. This platform is able to achieve sensitivity with the lowest clinical range (found in the infected patient) sample having 10² copies/mL of ZIKV target within 40 minutes making it a rapid ZIKV screening POC diagnostic method. This automated setup will facilitate accurate ZIKV diagnosis with low cost and high sensitivity, providing an excellent opportunity for field application and eliminating the requirement of benchtop examination in resource-limited areas.

### REFERENCES CITED

1. Bataille A, Cunningham AA, Cedeno V, et al. Evidence for regular ongoing introductions of mosquito disease vectors into the Galapagos Islands. Proc. R. Soc. B. 2009;276(1674):3769-3775.
2. Fajardo A, Cristina J, Moreno P. Emergence and spreading potential of Zika virus. Front Microbial. 2016; 7:1667.
3. Singh RK, Dhama K, Karthik: K, et al. Advances in diagnosis, surveillance, and monitoring of Zika virus: an update. Front Microbial. 2018; 8:2677.
4. Centers for Disease Control and Prevention. Yearly case counts for US states and territories.
https://www.cdc.gov/zika/reporting/index.html?CDC_AA_refVal=https%3A%2F% 2Fwww.cdc.gov%2Fzik:a%2Freporting%2Fcase-counts.html. Accessed October 03, 2019.
5. Grischott F, Puhan M, Hatz C, Schlagenhauf P. Non-vector-borne transmission of Zika virus: A systematic review. Travel Med Infect Di. 2016;14(4):313-330.
6. Kaarj K, Akarapipad P, Yoon J-Y. Simpler, faster, and sensitive Zika virus assay using smartphone detection of loop-mediated isothermal amplification on paper microfluidic chips. Sci. Rep. 2018;8(1):12438.
7. Herrada CA, Kabir M, Altamirano R, Asghar W. Advances in Diagnostic Methods for Zika Virus Infection. J. Med. Devices. 2018;12(4).
8. Kuno G, Chang G-J. Full-length sequencing and genomic characterization of Bagaza, Kedougou, and Zika viruses. Arch. Viral. 2007;152(4):687-696.
9. Lee D, Shin Y, Chung S, Hwang KS, Yoon DS, Lee JH. Simple and highly sensitive molecular diagnosis of Zik:a virus by lateral flow assays. Anal. Chem. 2016;88(24):12272-12278.
10. Lustig Y, Zelena H, Venturi G, et al. Sensitivity and kinetics of an NS1-based Zika virus enzyme-linked immunosorbent assay in Zika virus-infected travelers from Israel, the Czech Republic, Italy, Belgium, Germany, and Chile. J Clin Microbial. 2017;55(6):1894-1901.
11. L'Huillier AG, Hamid-Allie A, Kristjanson E, et al. Evaluation of Euroimmun anti-Zika virus IgM and IgG enzyme-linked immunosorbent assays for Zika virus serologic testing. J Clin Microbial. 2017;55(8):2462-2471.
12. Granger D, Hilgart H, Misner L, et al. Serologic testing for Zika virus: comparison of three Zika virus IgM-screening enzyme-linked immunosorbent assays and initial laboratory experiences. Clin Microbial Infect. 2017;55(7):2127-2136.
13. Gorchakov R, Berry RM, Patel SM, El Sahly HM, Ronca SE, Murray KO. Optimizing PCR detection of Zika virus from various body fluids. Am J Trop Med Hyg. 2019;100(2):427-433.
14. Viedma MdPM, Puri V, Oldfield LM, Shabman RS, Tan GS, Pickett BE. Optimization of qRT-PCR assay for zika virus detection in human serum and urine. Virus Res. 2019;263:173-178.
15. Schrader C, Schielke A, Ellerbroek L, Johne R. PCR inhibitors-occurrence, properties and removal. J. Appl. Microbial. 2012;113(5):1014-1026.
16. Notomi T, Okayama H, Masubuchi H, et al. Loop-mediated isothermal amplification of DNA. Nucleic Acids Res. 2000;28(12).
17. Drain PK, Hyle EP, Noubary F, et al. Diagnostic point-of-care tests in resource-limited settings. Lancet Infect Dis. 2014;14(3):239-249.
18. Velders AH, Schoen C, Saggiomo V. Loop-mediated isothermal amplification (LAMP) shield for Arduino DNA detection. BMC Res. 2018;11(1):93.
19. Song J, Mauk MG, Hackett BA, Cherry S, Bau HH, Liu C. Instrument-free point-of-care molecular detection of Zika virus. Anal. Chem. 2016;88(14):7289-7294.
20. Ganguli A, Omob A, Yu H, et al. Hands-free smartphone-based diagnostics for simultaneous detection of Zika, Chikungunya, and Dengue at point-of-care. Biomed. Microdevices. 2017;19(4):73.
21. Miyamoto S, Sano S, Takahashi K, Jikihara T. Method for colorimetric detection of double-stranded nucleic acid using leuco triphenylmethane dyes. Anal. Biochem. 2015; 473:28-33.
22. Roy S, Mohd-Nairn NF, Safavieh M, Ahmed MU. Colorimetric nucleic acid detection on paper microchip using loop mediated isothermal amplification and crystal violet dye. ACS Sens. 2017;2(11):1713-1720.
23. Song J, Liu C, Mauk MG, et al. Two-stage isothermal enzymatic amplification for concurrent multiplex molecular detection. Clin Chem. 2017;63(3):714-722.
24. National Center for Biotechnology Information. Virus Variation.
https://www.ncbi.nlm.nih.gov/genomesNirusVariation/Database/nph-select.cgi. Accessed December 18, 2019.
25. Clustal Omega. Multiple Sequence Alignment.
https://www.ebi.ac.uk/Tools/msa/clustalo/. Accessed December 18, 2019.
26. Faye O, Diallo D, Diallo M, Weidmann M. Quantitative real-time PCR detection of Zika virus and evaluation with field-caught mosquitoes. VirolJ. 2013;10(1):311.
27. Faye O, Dupressoir A, Weidmann M, Ndiaye M, Sall AA. One-step RT-PCR for detection of Zika virus. J Clin Virol. 2008;43(1):96-101.
28. Coarsey C, Coleman B, Kabir MA, Sher M, Asghar W. Development of a flow-free magnetic actuation platform for an automated microfluidic ELISA. RSC Adv. 2019;9 (15):8159-8168.
29. Kabir MA, Zilouchian H, Sher M, Asghar W. Development of a Flow-Free Automated Colorimetric Detection Assay Integrated with Smartphone for Zika NS1. Diagnostics. 2020;10(1):42.
30. Rappa K, Samargia J, Sher M, Pino JS, Rodriguez HF, Asghar W. Quantitative analysis of sperm rheotaxis using a microfluidic device. Microfluid Nanofluid. 2018;22(9):100.
31. Sher M, Asghar W. Development of a multiplex fully automated assay for rapid quantification of CD4+ T cells from whole blood. Biosens. Bioelectron. 2019; 142:111490.
32. Asghar W, Sher M, Khan NS, Vyas JM, Demirci U. Microfluidic chip for detection of fungal infections. ACS omega. 2019;4(4):7474-7481.
33. Chambers TJ, Hahn CS, Galler R, Rice CM. Flavivirus genome organization, expression, and replication. Annu Rev Microbiol. 1990;44(1):649-688.
34. Tisdale M. Monitoring of viral susceptibility: new challenges with the development of influenza NA inhibitors. J Med Virol. 2000;10(1):45-55.

1. An automated platform for the detection of a virus, bacteria, and/or other organism of interest from a clinical sample, said automated platform comprising:
a disposable microfluidic chip for receiving the clinical sample therethrough for isolating, purifying and amplifying the virus, bacteria, and/or other organism of interest in the clinical samples;
a magnetic actuation platform; said magnetic actuation platform being activated to convey the clinical samples through said disposable microfluidic chip; and
a surface heater for heating the clinical samples.

2. The automated platform of claim 1, wherein the disposable microfluidic chip comprises three layers comprising:
a top layer comprised of poly(methyl methacrylate) (PMMA) at a thickness of 750 μm;
a middle comprised of poly(methyl methacrylate) (PMMA) at a thickness of 1.5 mm; and
a bottom layer comprised of poly(methyl methacrylate) (PMMA) at a thickness of 750 μm.

3. The automated platform of claim 1, wherein the disposable microfluidic chip comprises plurality of independent aqueous chambers separated by a plurality of elliptical shaped valving chambers containing mineral oil which work as valves, the plurality of independent aqueous chambers comprising:
an inlet chamber;
at least one washing buffer chamber;
an amplification chamber; and
an unconnected oval-shaped sensor chamber.

4. The automated platform of claim 2, wherein the top layer of the disposable microfluidic chip contains two pipette inlets (0.4 mm diameter) above each chamber.

5. The automated platform of claim 4, wherein one inlet discharges the fluid into the disposable microfluidic chip and another inlet liberates the air out of the chamber.

6. The automated platform of claim 3, wherein the plurality of independent aqueous chambers comprises four chambers.

7. The automated platform of claim 3, wherein the at least one washing buffer chamber comprises a first washing buffer chamber and a second washing buffer chamber.

8. The automated platform of claim 3, wherein the plurality of elliptical shaped valving chambers comprises a first valving chamber, a second valving chamber, and a third valving chamber.

9. The automated platform of claim 3, wherein the unconnected oval-shaped sensor chamber is separated from the other chambers.

10. The automated platform of claim 1, wherein the magnetic actuation is executed by at least one small magnet which is enclosed with stepper motor and able to move bidirectionally on stepper motor linear slide rails.

11. The magnetic actuation platform of claim 10, wherein the stepper motor linear slide rails are connected to the stepper motor by a power output wire.

12. The automated platform of claim 1, wherein said platform comprises an in-built surface heater to control the temperature required for isothermal amplification; wherein said heater is activated at the start of the isothermal amplification procedure to a temperature of 70° C.±2° C.

13. The automated platform of claim 3, wherein the sensor chamber and the amplification chamber are filled with a reagent and the surface heater is attached to both chambers.

14. The automated platform of claim 10, wherein magnetic actuation is coordinated by an automated circuit board and the at least one magnetic bead movement from one chamber to another.

15. The automated platform of claim 1, wherein an automated circuit board controls the temperature of reagents enclosed in the amplification chamber of the microfluidic chip.

16. The automated platform of claim 1, wherein multiple samples can be tested simultaneously.

17. The automated platform of claim 1, wherein the virus to be tested is one selected from the group consisting of DENV, ZIKV, HIV, coronavirus, and HCV.

18. A method of isolation, purification, and amplification of a virus, bacteria, and/or other organism of interest from clinical samples using a Loop-Mediated Isothermal Amplification (“LAMP”) method on the automated platform of claim 1, wherein said LAMP method comprises
a. Obtaining human fluid samples containing concentrations of the virus, bacteria, and/or other organism of interest;
b. Filling an amplification chamber of the microfluidic chip with LAMP reagents, and sealing pipette inlets and a sensor chamber with epoxy glue;
c. Preparing a plasma sample for an inlet chamber of the microfluidic chip, adding at least one magnetic bead, and incubating;
d. Loading a first buffer into a first washing buffer chamber; and a second buffer into a second washing buffer chamber;
e. Adding mineral oil to an at least one and preferably three valving chambers;
f. Adding the plasma sample of step c into the inlet chamber of the microfluidic chip;
g. Attaching the surface heater on top of the amplification chamber and the sensor chamber so that the chambers are heated to the desired temperature before the magnetic bead is guided into the said chambers; and
h. Coordinating magnetic actuation by an automated circuit board, wherein the magnetic bead is moved and subsequently incubated from one chamber to the other to perform a LAMP analysis of the said plasma sample.


SEQUENCE LISTING (10 sequences)

SEQ ID NO: 1 - DNA, 16 bp, Artificial Sequence
  Synthetic construct
ctggttggttcacaag

SEQ ID NO: 2 - DNA, 15 bp, Artificial Sequence
  Synthetic construct
cagagctccagcaag

SEQ ID NO: 3 - DNA, 38 bp, Artificial Sequence
  Synthetic construct
gtgtggagttccggtgtctagtggttccacgacattcc

SEQ ID NO: 4 - DNA, 41 bp, Artificial Sequence
  Synthetic construct
ctggtagagttcaaggacgcatgctccttcttgactcccta

SEQ ID NO: 5 - DNA, 19 bp, Artificial Sequence
  Synthetic construct
cccagcgtgccaaggtaat

SEQ ID NO: 6 - DNA, 21 bp, Artificial Sequence
  Synthetic construct
catgccaaaaggcaaactgtc

SEQ ID NO: 7 - DNA, 63 bp, Artificial Sequence
  Synthetic construct
ggaagctgcaaacgcggtcgcaaacctggttggaatcaatcaagagaatacacaagcacttga

SEQ ID NO: 8 - DNA, 63 bp, Artificial Sequence
  Synthetic construct
ggaagctgcaaacgcggtcgcagacctggttggaatcaatcaagagaatatacaagcacctga

SEQ ID NO: 9 - DNA, 63 bp, Artificial Sequence
  Synthetic construct
ggaagctgcaagcgcggtcgcaaacctggttggaatcaatcaagagaatacacaagcacttga

SEQ ID NO: 10 - DNA, 63 bp, Artificial Sequence
  Synthetic construct
ggaagctacaaacgcggtcgcaaacctggttggaatcaatcaagagaatacacaagcacttga